(19) United States
(12) Patent Application Publication
(10) Pub. No.: US 2026/0354663 A1
(43) Pub. Date: Nov. 12, 2026

(54) ENDOSCOPE

(71) Applicant: FUJIFILM Corporation, Tokyo (JP)

(72) Inventor: Takashi HARADA, Kanagawa (JP)

(73) Assignee: FUJIFILM Corporation, Tokyo (JP)

(21) Appl. No.: 19/658,712

(22) Filed: Apr. 27, 2026

(30) Foreign Application Priority Data
May 9, 2025 (JP) 2025-078824

(51) Int. Cl. A61B 1/00; A61B 1/005; A61B 1/06

(52) U.S. Cl. CPC A61B 1/00087; A61B 1/00103; A61B 1/005; A61B 1/06

(57) ABSTRACT

An endoscope includes an insertion part that is to be inserted into a subject; a distal end portion that is disposed on a distal end side of the insertion part; and an elevator for a treatment tool that is disposed on the distal end portion, in which an outer diameter of the distal end portion is 7.0 mm or more and 10.3 mm or less, and an outer diameter of the insertion part is 7.4 mm or more and 10.3 mm or less.

### CROSS-REFERENCE TO RELATED APPLICATIONS

The present application claims priority under 35 U.S.C. §119 to Japanese Patent Application No. 2025-078824 filed on May 9, 2025. The above application is hereby expressly incorporated by reference, in its entirety, into the present application.

### BACKGROUND OF THE INVENTION

### 1. Field of the Invention

The disclosed technology relates to an endoscope.

### 2. Description of the Related Art

US2022/151480A discloses a disposable endoscope comprising an elevator that changes a delivery direction of a treatment tool on a distal end side of an insertion part.

JP2024-066413A discloses an endoscope comprising a distal end constituent member suitable for single use.

JP2020-137947A discloses a technology capable of preventing folding failure due to a foreign material such as a gallstone in an endoscope comprising an elevator that changes a delivery direction of a treatment tool on a distal end side of an insertion part.

WO2019/159507A discloses an endoscope that drives a movable member provided in a distal end portion of an insertion part with an operation wire.

### SUMMARY OF THE INVENTION

An endoscope according to an aspect of the disclosed technology comprises: an insertion part that is to be inserted into a subject; a distal end portion that is disposed on a distal end side of the insertion part; and an elevator for a treatment tool that is disposed on the distal end portion, in which an outer diameter of the distal end portion is 7.0 mm or more and 10.3 mm or less, and an outer diameter of the insertion part is 7.4 mm or more and 10.3 mm or less.

### BRIEF DESCRIPTION OF THE DRAWINGS

FIG. 1 is a schematic view showing a schematic configuration of an endoscope device 100 according to an aspect of the disclosed technology.

FIG. 2 is a schematic view showing an internal configuration of a single-use endoscope 40.

FIG. 3 is a perspective view showing a configuration example of a distal end portion 44 and a bendable part 43 of the single-use endoscope 40.

FIG. 4 is a perspective view showing the vicinity of the distal end portion 44 in a state in which a first support member 13 and a covering member 432 are omitted.

FIG. 5 is a perspective view showing the vicinity of the distal end portion 44 in a state in which the first support member 13 and the covering member 432 are omitted, the view being from a direction different from that of FIG. 4.

FIG. 6 is a perspective view showing the vicinity of the distal end portion 44 in a state in which the first support member 13 and the covering member 432 are omitted, the view being from a direction different from those of FIGS. 4 and 5.

FIG. 7 is an exploded perspective view (part 1) of the distal end portion 44.

FIG. 8 is an exploded perspective view (part 2) of the distal end portion 44.

FIG. 9 is a perspective view showing a first constituent member 51 as viewed from a distal end side.

FIG. 10 is a perspective view showing the first constituent member 51 as viewed from a proximal end side.

FIG. 11 is a plan view showing the first constituent member 51 as viewed from the proximal end side.

FIG. 12 is an exploded perspective view of a third constituent member 53, a fourth constituent member 54, and a structure disposed between the third constituent member 53 and the fourth constituent member 54.

FIG. 13 is an exploded perspective view of the third constituent member 53, the fourth constituent member 54, and the structure disposed between the third constituent member 53 and the fourth constituent member 54, the view being from a direction different from that of FIG. 12.

FIG. 14 is a perspective view showing a configuration of a second constituent member 52 and an elevator 80 supported by the second constituent member 52.

FIG. 15 is an exploded perspective view of the configuration shown in FIG. 14.

FIG. 16 is a schematic view of a cross section taken through a through-hole into which a pivot shaft of the elevator 80 is inserted in FIG. 14.

FIG. 17 is a schematic view of a cross section of the distal end portion 44 taken perpendicular to a left-right direction, and is a view showing a state in which the elevator 80 is in a folded position.

FIG. 18 is a schematic view of a cross section of the distal end portion 44 taken perpendicular to the left-right direction, and is a view showing a state in which the elevator 80 is in an intermediate upright position.

FIG. 19 is a schematic view of a cross section of the distal end portion 44 taken perpendicular to a longitudinal direction thereof.

### DESCRIPTION OF THE PREFERRED EMBODIMENTS

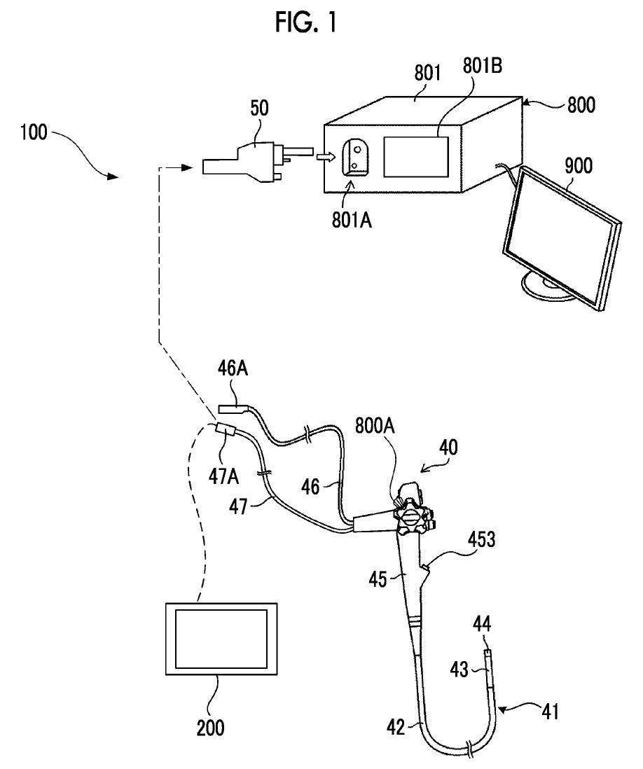

FIG. 1 is a schematic view showing a schematic configuration of an endoscope device 100 according to an aspect of the disclosed technology. As shown in FIG. 1, the endoscope device 100 comprises a control device 800 capable of controlling a reusable endoscope that can be used repeatedly (a plurality of times) by a reprocessing (disinfection and cleaning) treatment, a display device 900 such as a liquid-crystal display or an organic electro-luminescence (EL) display, a single-use endoscope 40 that can be used once (one-time use), and an adapter 50 that relays a connection between the single-use endoscope 40 and the control device 800.

The control device 800 includes an input device 801B (a physical button, a touch panel, a keyboard, a mouse, or the like) that receives an input from a user, and integrally controls the endoscope device 100 in response to an instruction input from the input device 801B.

The control device 800 is configured to connect to the reusable endoscope and control the connected reusable endoscope. The control device 800 is configured to connect to the single-use endoscope 40 via the adapter 50 and control the single-use endoscope 40 via the adapter 50. The single-use endoscope 40 can be used by being connected to a portable tablet terminal 200 in addition to the control device 800. The single-use endoscope 40 can be controlled by the tablet terminal 200 in a case where a connector 47A is connected to the tablet terminal 200. In addition, the tablet terminal 200 is capable of generating an image and displaying the image.

The connection between the endoscope and the control device includes at least one of electrical connection between the endoscope and the control device (a state in which transmission and reception of signals or transmission and reception of power can be performed by an electric wire, radio waves, light, or the like), optical connection between the endoscope and the control device (a state in which illumination light can be transmitted and received), or spatial connection between the endoscope and the control device (a state in which transmission and reception of a gas such as air can be performed).

The single-use endoscope 40 is exemplified by a flexible scope, and comprises a flexible insertion part 41 that is to be inserted into a subject, an operation unit 45 that is disposed on a proximal end side of the insertion part 41 and that is capable of operating the insertion part 41, a first cord part 46 that is provided to extend from the operation unit 45, a connector 46A that is provided at an end portion of the first cord part 46 and that is attachably and detachably connected to the adapter 50, a second cord part 47 that is provided to extend from the operation unit 45, and a connector 47A that is provided at an end portion of the second cord part 47 and that is attachably and detachably connected to the adapter 50. The connector 46A is configured not to be electrically connected to the adapter 50. The connector 47A is configured to be electrically connected to the adapter 50.

The single-use endoscope 40 is not limited to the flexible scope, and may be another type of endoscope, such as a rigid scope. The single-use endoscope 40 is a duodenoscope having an elevator for a treatment tool capable of changing a lead-out angle of the treatment tool, but is not limited thereto. Each of the first cord part 46 and the second cord part 47 comprises a tubular outer sheath and a member that is inserted and disposed inside the outer sheath.

The insertion part 41 has a distal end portion 44 that is disposed on a distal end side, a bendable part 43 that is disposed on a proximal end side of the distal end portion 44 and that is capable of performing a bending operation, and a flexible part 42 that connects the bendable part 43 and the operation unit 45.

The bendable part 43 is formed by connecting a plurality of bendable pieces (angle rings), and is configured to bend in a predetermined direction (four directions in the present embodiment) in response to an operation of an angle knob provided in the operation unit 45. The flexible part 42 is elongated and long, and has flexibility.

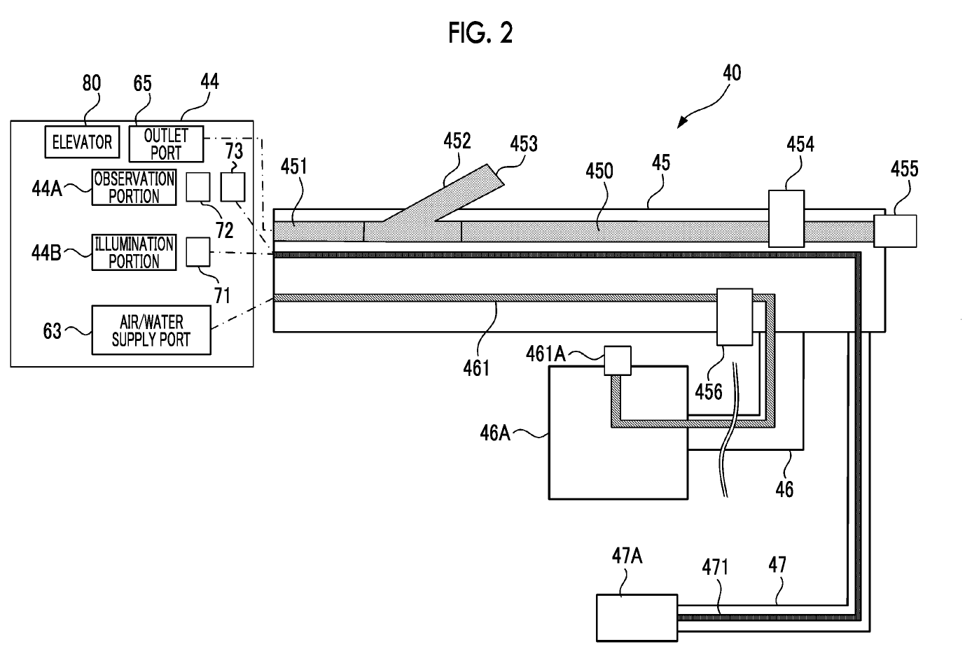

FIG. 2 is a schematic view showing an internal configuration of the single-use endoscope 40. As shown in FIG. 2, the distal end portion 44 of the single-use endoscope 40 is provided with an illumination portion 44B that emits illumination light, an observation portion 44A on which observation light, which is reflected light obtained by reflection of the illumination light from the illumination portion 44B by a subject, is incident, a treatment tool outlet port 65, an elevator 80 that is capable of changing a direction of a treatment tool led out from the outlet port 65, and an air/water supply port (nozzle) 63. The illumination portion 44B and the observation portion 44A are provided on an outer peripheral surface of the distal end portion 44, and the single-use endoscope 40 is of a so-called side-viewing type.

The air/water supply port 63 is configured to deliver any of air (gas) and water (liquid) toward the observation portion 44A. The observation portion 44A can be cleaned by the water delivered from the air/water supply port 63. Water droplets adhering to the observation portion 44A can be removed by the air delivered from the air/water supply port 63.

The connector 46A of the first cord part 46 is provided with an air/water supply connection fitting 461A to which an air/water supply device can be connected.

An air/water supply pipe line 461 as a liquid pipe line and a gas pipe line that communicates with the air/water supply port 63 is provided inside the insertion part 41. The air/water supply pipe line 461 includes, for example, a rigid pipe provided at the distal end portion 44 of the insertion part 41 and a flexible tube connected to the rigid pipe. The air/water supply pipe line 461 is connected to the air/water supply connection fitting 461A of the connector 46A from the air/water supply port 63 through the inside of the insertion part 41, the operation unit 45, and the first cord part 46.

In a case where the air/water supply device including an air supply pump or a water supply tank connected to the air/water supply connection fitting 461A is operated, air or water can be supplied to the air/water supply port 63 through the air/water supply pipe line 461. A valve mechanism 456 is provided inside the operation unit 45 at an intermediate portion of the air/water supply pipe line 461. The valve mechanism 456 is configured as, for example, an air/water supply button. By opening and closing a valve provided in the valve mechanism 456, it is possible to switch on and off the supply of air or water from the air/water supply device connected to the air/water supply connection fitting 461A to the air/water supply port 63.

A treatment tool pipe line 451 communicating with the outlet port 65 is disposed inside the insertion part 41. The treatment tool pipe line 451 is connected to a branch member 452 inside the operation unit 45. The branch member 452 is a member that branches the treatment tool pipe line 451 into two pipe lines, and an outlet of one of the two pipe lines is a treatment tool inlet port 453 of the operation unit 45.

An operation unit pipe line 450 is connected to an outlet of the other one of the two pipe lines in the branch member 452, and a suction nozzle 455 is connected to the operation unit pipe line 450. The suction nozzle 455 protrudes from a housing of the operation unit 45 and can be connected to a suction device (not shown). A valve mechanism 454 is provided at an intermediate portion of the operation unit pipe line 450. The valve mechanism 454 is configured as, for example, a suction button.

By opening and closing a valve provided in the valve mechanism 454, a negative pressure can be generated by suction of air inside the treatment tool pipe line 451, the branch member 452, and the operation unit pipe line 450 from the suction device connected to the suction nozzle 455. As a result, body fluids or the like in a subject can be suctioned from the outlet port 65 via the treatment tool pipe line 451. As described above, the outlet port 65 also functions as a suction port provided on the distal end side of the insertion part 41, and the treatment tool pipe line 451 also functions as a suction pipe line provided to extend from the suction port to the operation unit 45.

The distal end portion 44 of the insertion part 41 is provided with an imaging optical system 72 that forms, as an optical image, subject light from an observation target site incident on the observation portion 44A, an imaging element 73 that converts the optical image formed by the imaging optical system 72 into an image signal, in other words, that images a subject through the observation portion 44A, and a light emitting unit 71 that generates illumination light emitted from the illumination portion 44B. As described above, the observation portion 44A and the illumination portion 44B are provided on a side surface of the distal end portion 44. The light emitting unit 71 includes a light emitting element composed of a semiconductor device such as a light emitting diode. By using the light emitting element included in the light emitting unit 71 as a semiconductor device, the manufacturing costs of the single-use endoscope 40 can be reduced.

A signal line 471 connected to the imaging element 73 and the light emitting unit 71 is provided inside the insertion part 41. The signal line 471 is connected to a connector 47A from the distal end portion 44 through the inside of the insertion part 41, the operation unit 45, and the second cord part 47. In a case where the connector 47A is connected to the adapter 50, and the adapter 50 is connected to a connector connection portion 801A of the control device 800, the control device 800 and the single-use endoscope 40 are electrically connected to each other. As a result, the control device 800 is capable of controlling the single-use endoscope 40 and processing the imaging signal acquired from the single-use endoscope 40.

In the above description, a configuration in which the adapter 50 and the connector 46A are separable from each other has been described, but the adapter 50 and the connector 46A may be integrated with each other.

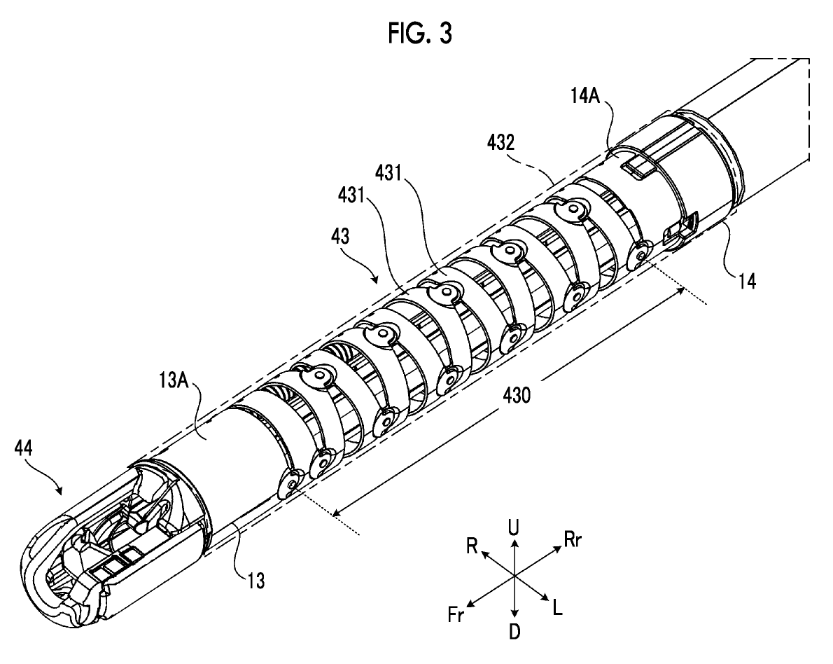

FIG. 3 is a perspective view showing a configuration example of the distal end portion 44 and the bendable part 43 of the single-use endoscope 40. In FIG. 3, a tubular covering member 432 constituting an outer surface of the bendable part 43 is shown by a one-dot chain line.

FIG. 3 shows a distal end direction Fr from the proximal end side toward the distal end side and a proximal end direction Rr from the distal end side toward the proximal end side as a longitudinal direction of the insertion part 41 (hereinafter, simply referred to as a longitudinal direction). In addition, FIG. 3 shows an up direction U, a down direction D, a right direction R, and a left direction L among four directions in which a bending operation of the bendable part 43 is possible.

The up direction U and the down direction D are orthogonal to the longitudinal direction (the distal end direction Fr and the proximal end direction Rr) of the insertion part 41. The right direction R and the left direction L are orthogonal to the longitudinal direction of the insertion part 41. The up direction U and the down direction D are orthogonal to the right direction R and the left direction L. The up direction U and the down direction D are collectively referred to as an up-down direction. The left direction L and the right direction R are collectively referred to as a left-right direction.

As shown in FIG. 3, the bendable part 43 comprises a structure 430 that is bendable in four directions by a bending operation and that is formed of a plurality of annular bending pieces 431 arranged in the longitudinal direction and rotatably connected to each other, a tubular first support member 13 that supports a distal end side of the structure 430, a tubular second support member 14 that supports a proximal end side of the structure 430, and a tubular covering member 432 that directly covers the structure 430, the first support member 13, and the second support member 14. The bending piece 431 constitutes an annular member. The first support member 13 is disposed on a distal end side of the bending piece 431 on the most distal end side of the structure 430 and rotatably supports the bending piece 431. A proximal end side of the distal end portion 44 is inserted into the first support member 13 on a distal end side thereof.

The second support member 14 is disposed on a proximal end side of the bending piece 431 on the most proximal end side of the structure 430 and rotatably supports the bending piece 431. A distal end side of the flexible part 42 is inserted into the second support member 14 on a proximal end side thereof.

Four operation wires for performing the bending operation of the structure 430 are fixed inside the first support member 13. The four operation wires extend to the inside of the operation unit 45 through the inside of the insertion part 41 and are connected to an angle knob of the operation unit 45. A tubular body is constituted by the structure 430, the first support member 13, and the second support member 14. The covering member 432 constitutes a covering portion that directly covers the tubular body.

It is preferable that the covering member 432 is made of a non-stretchable material. The structure 430, the first support member 13, and the second support member 14 are made of a resin, so that the manufacturing costs of the single-use endoscope 40 can be reduced. The covering member 432 can be made of, for example, an elastomer, thermoplastic polyurethane, or the like.

A distal end side of the covering member 432 is fixed to an outer peripheral surface 13A of the first support member 13 by an adhesive. A proximal end side of the covering member 432 is fixed to an outer peripheral surface 14A of the second support member 14 by an adhesive. It is preferable that the covering member 432 is configured such that, both in a state in which the structure 430 extends linearly and in a state in which the structure 430 is bent to a maximum extent, a first portion of the covering member 432 between the outer peripheral surface 13A and the outer peripheral surface 14A has slack. It is preferable that the slack occurs at a plurality of locations in the longitudinal direction.

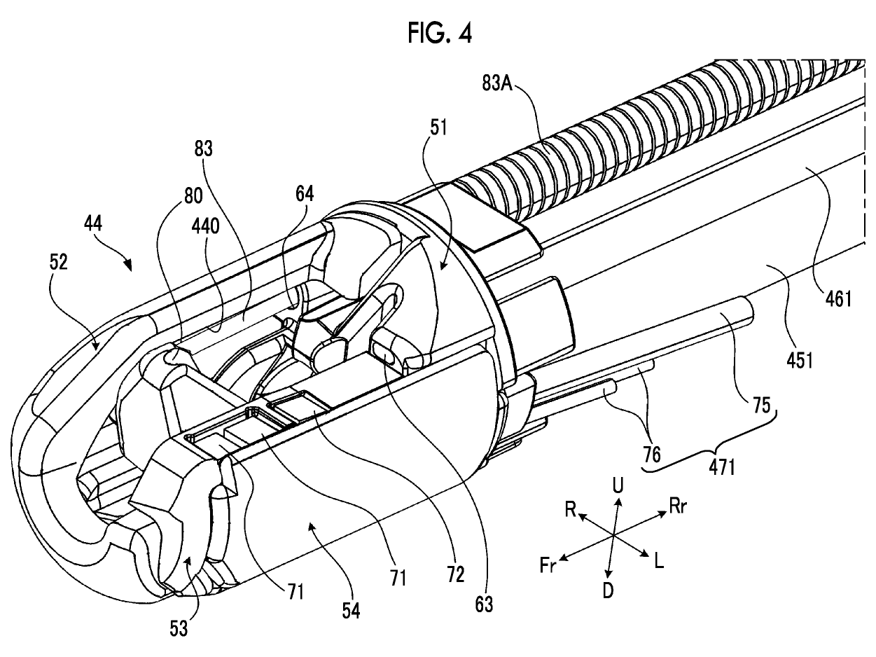

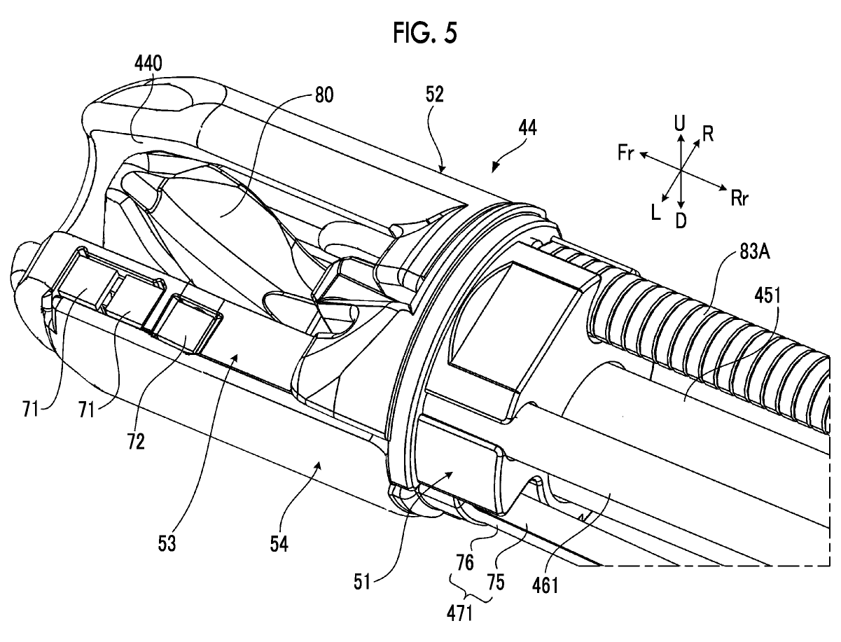

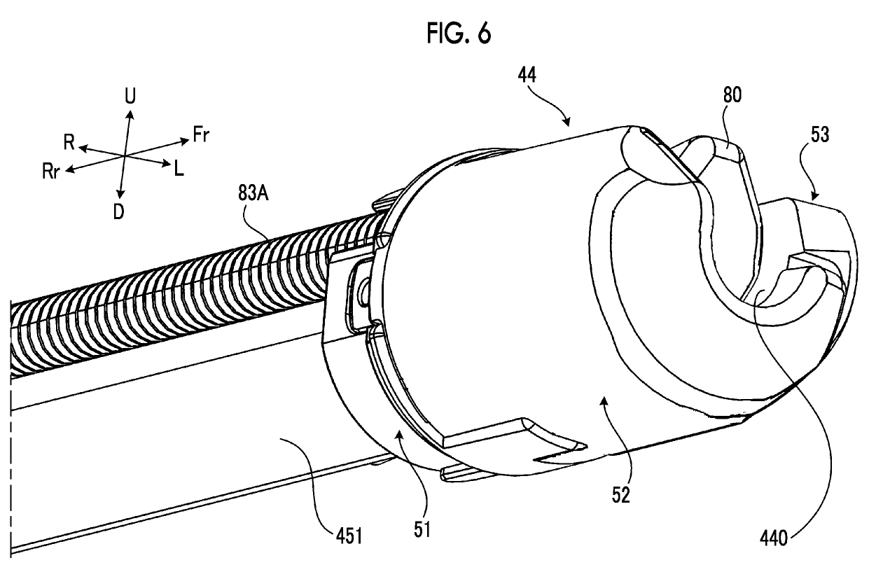

FIG. 4 is a perspective view showing the vicinity of the distal end portion 44 in a state in which the first support member 13 and the covering member 432 are omitted. FIG. 5 is a perspective view showing the vicinity of the distal end portion 44 in a state in which the first support member 13 and the covering member 432 are omitted, as viewed from a direction different from that of FIG. 4. FIG. 6 is a perspective view showing the vicinity of the distal end portion 44 in a state in which the first support member 13 and the covering member 432 are omitted, as viewed from a direction different from those of FIGS. 4 and 5.

As shown in FIG. 4, an accommodation recessed portion 440 that is open to a distal end side and an upper side is formed substantially at the center of the distal end portion 44 in the left-right direction. An elevator 80 for a treatment tool is disposed in the accommodation recessed portion 440 to be movable in the up-down direction. A distal end side of an operation wire 83 for operating the elevator 80 is disposed in the accommodation recessed portion 440, and a distal end of the operation wire 83 is fixed to the elevator 80. Since the elevator 80 is made to contain a resin material, the manufacturing costs can be reduced. As the resin material used for the elevator 80, for example, polyether ether ketone (PEEK) can be used. The elevator 80 may be entirely made of a resin, or may be an insert-molded product using glass, metal, or the like.

On an outer peripheral side of the distal end portion 44, two light emitting units 71 and an imaging optical system 72 are arranged in the longitudinal direction on an upper surface of a left-side portion of the accommodation recessed portion 440. The two light emitting units 71 and the imaging optical system 72 are covered with a transparent resin (not shown). The illumination portion 44B includes a region of the transparent resin facing the light emitting units 71. The observation portion 44A includes a region of the transparent resin facing the imaging optical system 72. As described above, the single-use endoscope 40 is configured as a side-viewing endoscope.

As shown in FIG. 4, the air/water supply port 63 is disposed on the outer peripheral surface of the distal end portion 44 on an extension line connecting the light emitting unit 71 and the imaging optical system 72.

The distal end portion 44 includes a plurality of members. In the present embodiment, the plurality of members constituting the distal end portion 44 are four members including a first constituent member 51, a second constituent member 52 constituting a first member and disposed on a distal end side of the first constituent member 51, a third constituent member 53 disposed on the distal end side of the first constituent member 51, and a fourth constituent member 54 disposed on an outer peripheral side of the third constituent member 53 (outer peripheral side of the distal end portion 44).

Since each of the plurality of members constituting the distal end portion 44 is made to contain a resin material, the manufacturing costs can be reduced. As the resin material used for the distal end portion 44, for example, polyether ether ketone can be used.

At least one of the plurality of members constituting the distal end portion 44 may be configured to be radiopaque. For example, at least one of the plurality of members constituting the distal end portion 44 may include an insert-molded body including a radiopaque structure such as metal, or may include a filler having X-ray shielding properties. As the filler, for example, barium sulfate can be employed. The content of the filler in the member is preferably 51% or less.

The first constituent member 51 and the second constituent member 52 are engaged with each other in the longitudinal direction. The first constituent member 51 and the third constituent member 53 are engaged with each other in the longitudinal direction. The second constituent member 52 and the third constituent member 53 are engaged with each other in the longitudinal direction. The third constituent member 53 and the fourth constituent member 54 are engaged with each other in the left-right direction, which is a direction orthogonal to the longitudinal direction. The fourth constituent member 54 and the first constituent member 51 are engaged with each other in the left-right direction.

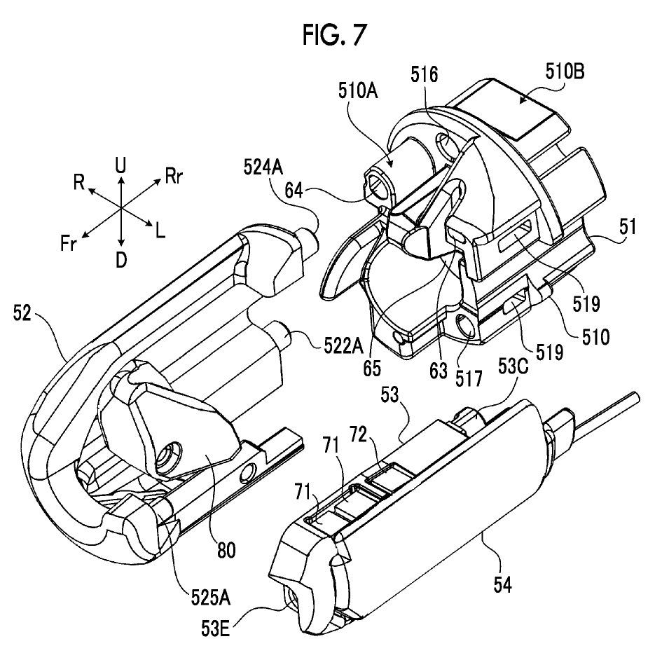

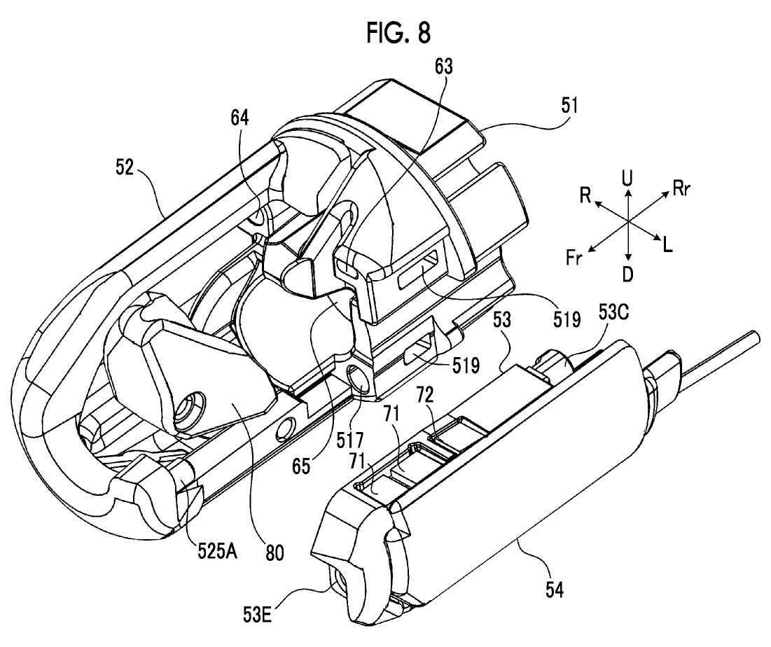

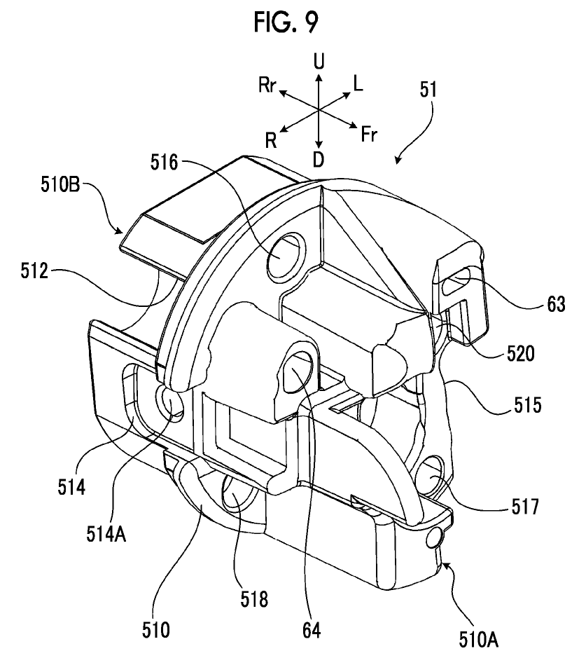

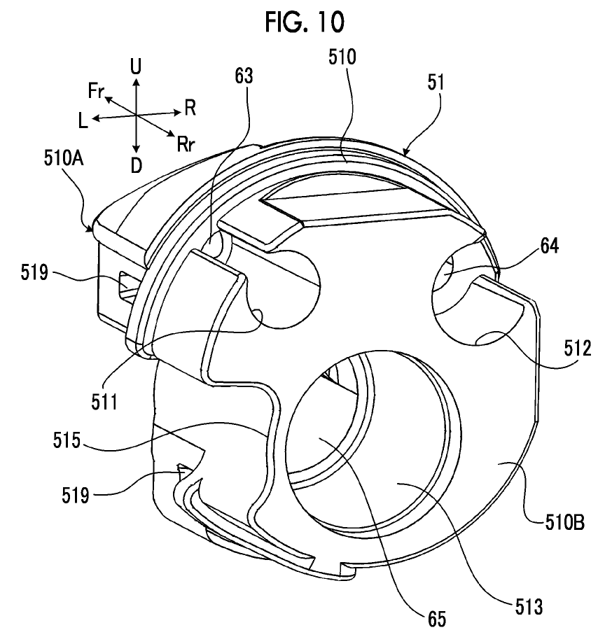

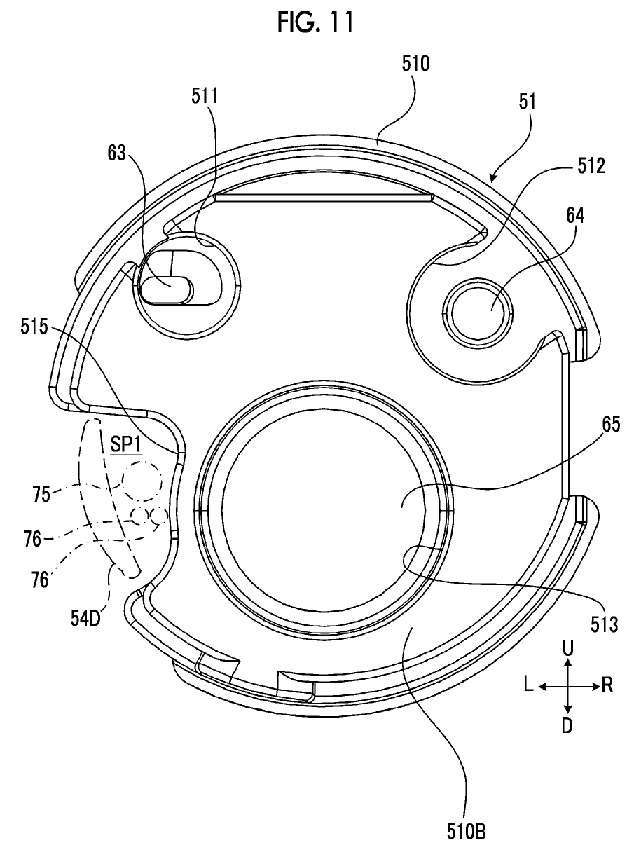

FIG. 7 is an exploded perspective view (part 1) of the distal end portion 44. FIG. 7 shows a state in which the second constituent member 52 and the third constituent member 53 are engaged with each other. FIG. 8 is an exploded perspective view (part 2) of the distal end portion 44. FIG. 8 shows a state in which the first constituent member 51 and the second constituent member 52 are engaged with each other. FIG. 9 is a perspective view of the first constituent member 51 as viewed from the distal end side. FIG. 10 is a perspective view of the first constituent member 51 as viewed from the proximal end side. FIG. 11 is a plan view of the first constituent member 51 as viewed from the proximal end side.

As shown in FIGS. 9-11, the first constituent member 51 has a disk-shaped base portion 510 perpendicular to the longitudinal direction. As shown in FIGS. 7 and 9, the first constituent member 51 is provided with a distal end-side protruding portion 510A that protrudes from the base portion 510 to the distal end side. The distal end-side protruding portion 510A and the base portion 510 are provided with an outlet port 65 constituted by a through-hole that extends in the longitudinal direction, at a lower side and substantially at the center of left and right sides. The distal end-side protruding portion 510A and the base portion 510 are provided with an air/water supply port 63 constituted by a through-hole that extends in the longitudinal direction, at a position above and to the left of the outlet port 65. The distal end-side protruding portion 510A and the base portion 510 are provided with an operation wire insertion hole port 64 constituted by a through-hole that extends in the longitudinal direction, at a position above and to the right of the outlet port 65.

As shown in FIGS. 7 and 10, the first constituent member 51 is provided with a proximal end-side protruding portion 510B that protrudes from the base portion 510 to the proximal end side. The proximal end-side protruding portion 510B is a portion inserted into the first support member 13. The proximal end-side protruding portion 510B is provided with a through-hole 513 that extends in the longitudinal direction and that communicates with the outlet port 65. The distal end side of the tube constituting the treatment tool pipe line 451 is inserted into and fixed to the through-hole 513, so that the treatment tool pipe line 451 and the outlet port 65 communicate with each other.

As shown in FIGS. 10 and 11, a cutout 511 that exposes a proximal end side of the air/water supply port 63, a cutout 512 that exposes a proximal end side of the operation wire insertion hole port 64, and a cutout 515 that is disposed on a left side of the through-hole 513 are formed in the proximal end-side protruding portion 510B. As shown in FIG. 11, the cutout 515 is closed by an extension region 54D of the fourth constituent member 54, so that a space SP1 in which the signal line 471 can be accommodated is formed.

The distal end side of the tube constituting the air/water supply pipe line 461 is inserted into the cutout 511, so that the air/water supply pipe line 461 and the air/water supply port 63 communicate with each other. The distal end side of a wire covering member 83A shown in FIG. 4 is inserted into the cutout 512. The operation wire 83 is inserted into the operation wire insertion hole port 64, and the operation wire 83 extending from the operation wire insertion hole port 64 to the proximal end side is inserted into the wire covering member 83A. The operation wire 83 and the wire covering member 83A that covers the operation wire 83 extend through the insertion part 41 to the operation unit 45. The operation wire 83 is connected to an operation lever 800A shown in FIG. 1, which is provided in the operation unit 45, and the elevator 80 can be operated by operating the operation lever 800A.

As shown in FIG. 9, an engagement hole portion 516 and an engagement hole portion 518 that constitute an engagement portion that is engageable with the second constituent member 52, and an engagement hole portion 517 and an engagement hole portion 520 that constitute an engagement portion that is engageable with the third constituent member 53 are provided on a surface of the distal end-side protruding portion 510A on the distal end side. Each of the engagement hole portion 516, the engagement hole portion 517, the engagement hole portion 518, and the engagement hole portion 520 includes, for example, a recessed portion that is recessed on the proximal end side.

As shown in FIG. 9, a receiving recessed portion 514 capable of receiving a part of the second constituent member 52 is provided on an outer peripheral surface of the proximal end-side protruding portion 510B on a right side. A hole portion 514A is provided in the receiving recessed portion 514.

As shown in FIGS. 7 and 10, two engagement hole portions 519 that constitute an engagement portion that is engageable with the fourth constituent member 54 in the left-right direction are provided on an outer peripheral surface of the distal end-side protruding portion 510A on the left side, and are spaced apart from each other in the up-down direction. The engagement hole portion 519 includes, for example, a recessed portion that is recessed on the right side.

As shown in FIG. 7, the second constituent member 52 is provided with an insertion projection portion 524A that is inserted into the engagement hole portion 516 of the first constituent member 51 and an insertion projection portion 522A that is inserted into the engagement hole portion 518 of the first constituent member 51. Each of the insertion projection portion 522A and the insertion projection portion 524A includes a protruding portion that protrudes to the proximal end side. The insertion projection portion 524A is inserted into the engagement hole portion 516, and the insertion projection portion 522A is inserted into the engagement hole portion 518, so that the first constituent member 51 and the second constituent member 52 engage with each other in the longitudinal direction. Each of a set of the engagement hole portion 516 and the insertion projection portion 524A and a set of the engagement hole portion 518 and the insertion projection portion 522A constitutes a first engagement portion.

As shown in FIG. 7, an engagement hole portion 53E that constitutes an engagement portion that is engageable with the second constituent member 52 in the longitudinal direction is provided on a surface of the third constituent member 53 on the distal end side. The engagement hole portion 53E includes a recessed portion that is recessed on the proximal end side. The second constituent member 52 is provided with an insertion projection portion 525A that is inserted into the engagement hole portion 53E. The insertion projection portion 525A includes a projection portion that projects to the proximal end side. The insertion projection portion 525A is inserted into the engagement hole portion 53E, so that the second constituent member 52 and the third constituent member 53 engage with each other in the longitudinal direction. A set of the engagement hole portion 53E and the insertion projection portion 525A constitutes a third engagement portion.

As shown in FIG. 7, an insertion projection portion 53C that is inserted into the engagement hole portion 520 of the first constituent member 51 is provided on a surface of the third constituent member 53 on the proximal end side. The insertion projection portion 53C includes a projection portion that projects to the proximal end side. The insertion projection portion 53C is inserted into the engagement hole portion 520, so that the first constituent member 51 and the third constituent member 53 engage with each other in the longitudinal direction. A set of the engagement hole portion 520 and the insertion projection portion 53C constitutes a second engagement portion.

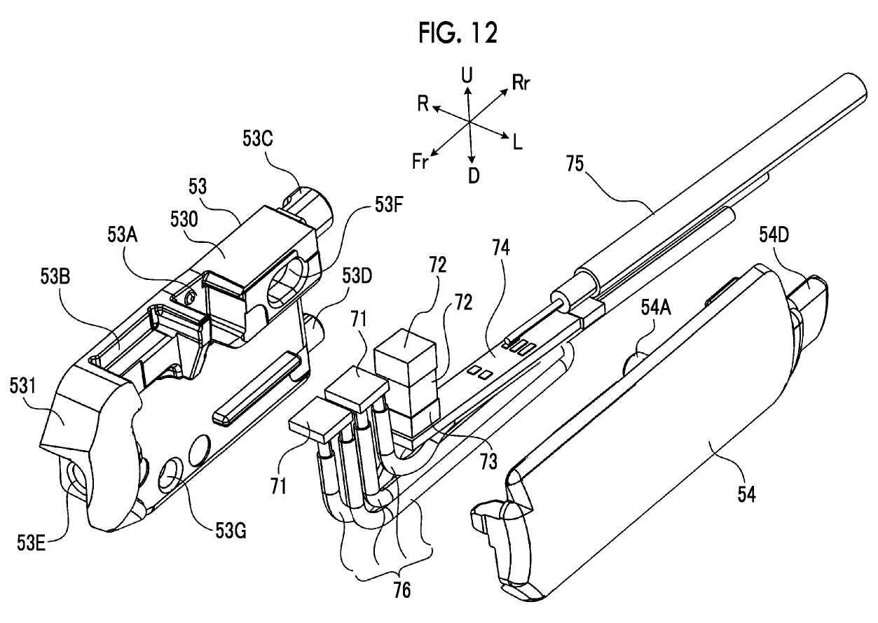

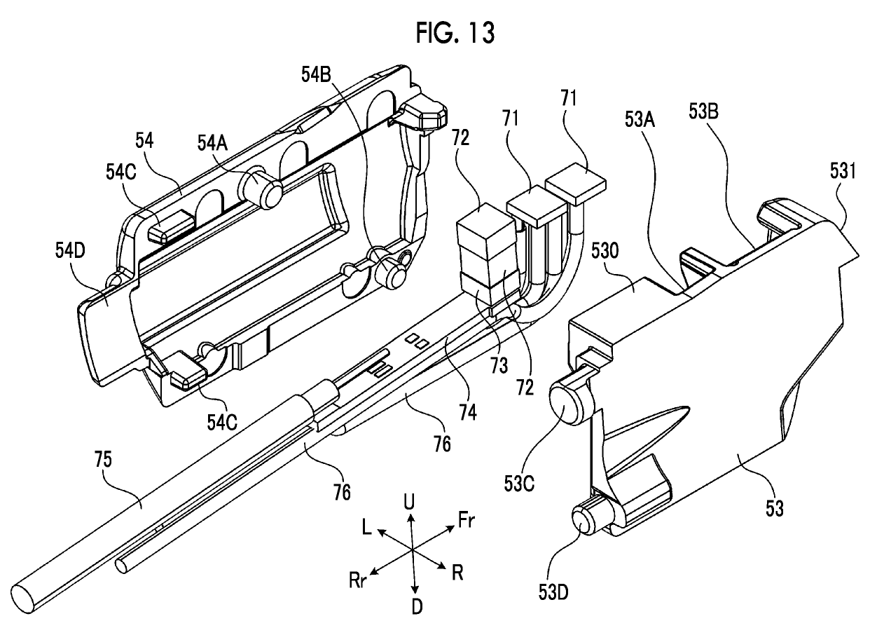

FIG. 12 is an exploded perspective view of the third constituent member 53 and the fourth constituent member 54, and a structure disposed between the third constituent member 53 and the fourth constituent member 54. FIG. 13 is an exploded perspective view of the third constituent member 53 and the fourth constituent member 54, and a structure disposed between the third constituent member 53 and the fourth constituent member 54, as viewed from a direction different from that of FIG. 12.

As shown in FIGS. 12 and 13, the third constituent member 53 has a rectangular plate-shaped base portion that intersects the left-right direction and that extends in the longitudinal direction, and has an upper surface wall portion 530 that rises to the left from an upper end portion of the base portion, and a distal end wall portion 531 that rises to the left from a distal end-side end portion of the base portion.

A recessed portion 53A and a recessed portion 53B that are recessed to the right side are formed in the upper surface wall portion 530. Two light emitting units 71 and a signal line 76 connected to the light emitting units 71 are disposed in the recessed portion 53B. The imaging optical system 72 is disposed in the recessed portion 53A. The above-described engagement hole portion 53E is formed in the distal end wall portion 531.

In a region surrounded by the upper surface wall portion 530 and the distal end wall portion 531 of the third constituent member 53, the signal line 76 connected to the light emitting units 71, the imaging element 73, the substrate 74 connected to the imaging element 73, and the signal line 75 connected to the substrate 74 are disposed. The signal line 471 shown in FIG. 2 is constituted by the signal line 75 and the signal line 76.

A surface of the third constituent member 53 on the proximal end side is provided with the above-described insertion projection portion 53C and an insertion projection portion 53D that is inserted into the engagement hole portion 517 of the first constituent member 51. The insertion projection portion 53D includes a projection portion that projects to the proximal end side. The insertion projection portion 53D is inserted into the engagement hole portion 517, so that the first constituent member 51 and the third constituent member 53 are engaged with each other in the longitudinal direction. A set of the engagement hole portion 517 and the insertion projection portion 53D constitutes a second engagement portion.

A surface of the third constituent member 53 on the left side is provided with an engagement hole portion 53F and an engagement hole portion 53G that constitute an engagement portion that engages with the fourth constituent member 54 in the left-right direction. Each of the engagement hole portion 53F and the engagement hole portion 53G is formed by a recessed portion that is recessed to the right side.

The fourth constituent member 54 is connected to the third constituent member 53, covers a surface of the third constituent member 53 on the right side and constitutes the outer peripheral surface of the distal end portion 44, and has a curved plate shape. As shown in FIG. 13, the fourth constituent member 54 is provided with an insertion projection portion 54A that is inserted into the engagement hole portion 53F and an insertion projection portion 54B that is inserted into the engagement hole portion 53G on a surface on a third constituent member 53 side. Each of the insertion projection portion 54A and the insertion projection portion 54B includes a projection portion that projects to the right side.

The insertion projection portion 54A is inserted into the engagement hole portion 53F, so that the third constituent member 53 and the fourth constituent member 54 are engaged with each other in the left-right direction. The insertion projection portion 54B is inserted into the engagement hole portion 53G, so that the third constituent member 53 and the fourth constituent member 54 are engaged with each other in the left-right direction. A set of the engagement hole portion 53F and the insertion projection portion 54A and a set of the engagement hole portion 53G and the insertion projection portion 54B each constitute an engagement portion that engages the third constituent member 53 and the fourth constituent member 54 with each other in a direction orthogonal to the longitudinal direction.

In a state in which the third constituent member 53 and the fourth constituent member 54 are engaged with each other, a part of the signal line 76, the substrate 74, and a part of the signal line 75 are disposed between the third constituent member 53 and the fourth constituent member 54.

As shown in FIGS. 12 and 13, the fourth constituent member 54 has an extension region 54D that extends from a proximal end to a proximal end side and that overlaps the cutout 515 of the first constituent member 51 in the direction perpendicular to the longitudinal direction.

As shown in FIG. 11, the extension region 54D is disposed at a position that covers the cutout 515. A space SP1 is formed between the extension region 54D and an inner peripheral surface of the cutout 515, and the signal line 75 and the signal line 76 can be disposed in the space SP1. According to this configuration, the first constituent member 51 can be made thinner than in a configuration in which the signal line 75 and the signal line 76 penetrate the inside of the first constituent member 51 and are inserted into the first support member 13.

As shown in FIG. 13, the fourth constituent member 54 is provided with an insertion projection portion 54C that is inserted into each of two engagement hole portions 519 of the first constituent member 51 on a surface on the third constituent member 53 side. The insertion projection portion 54C includes a projection portion that projects to the right side. The insertion projection portion 54C is inserted into the engagement hole portion 519, so that the first constituent member 51 and the fourth constituent member 54 are engaged with each other in the left-right direction. A set of the engagement hole portion 519 and the insertion projection portion 54C constitutes an engagement portion that engages the first constituent member 51 and the fourth constituent member 54 with each other in the left-right direction.

The distal end portion 44 can be assembled, for example, as follows. The observation module including the light emitting unit 71, the imaging optical system 72, the imaging element 73, the substrate 74, the signal line 75, and the signal line 76 is fixed to the third constituent member 53, and the fourth constituent member 54 is fixed to the third constituent member 53 to form a unit. In addition, an elevator unit including the elevator 80 is attached to the second constituent member 52.

Next, the third constituent member 53 is engaged with the second constituent member 52 in the longitudinal direction, and the assembly of the second constituent member 52 and the third constituent member 53 is engaged with the first constituent member 51 in the longitudinal direction. In this case, the engagement portions may be adhered to each other by an adhesive injected into the engagement portions.

As described above, the distal end portion 44 can be easily assembled by engagement of members in the longitudinal direction and a direction orthogonal to the longitudinal direction, and the manufacturing costs can be reduced. Since the third constituent member 53 is sandwiched between the first constituent member 51 and the second constituent member 52, an engaged state of the three constituent members can be firmly maintained.

Since the observation module is attached to the third constituent member 53 in a state in which the fourth constituent member 54 is not provided, the attachment of the observation module is easy. In addition, the observation module, the fourth constituent member 54, and the third constituent member 53 can be unitized in advance. The extension region 54D is inserted into the first support member 13 together with the proximal end-side protruding portion 510B of the first constituent member 51, and the movement in a radial direction is restricted by the first support member 13. Therefore, the engaged state of the distal end portion 44 can be firmly maintained. Since the fourth constituent member 54 is engaged with not only the third constituent member 53 but also the first constituent member 51, the movement of the first constituent member 51 in the longitudinal direction can be restricted, and the engaged state of the first constituent member 51, the second constituent member 52, and the third constituent member 53 can be firmly maintained.

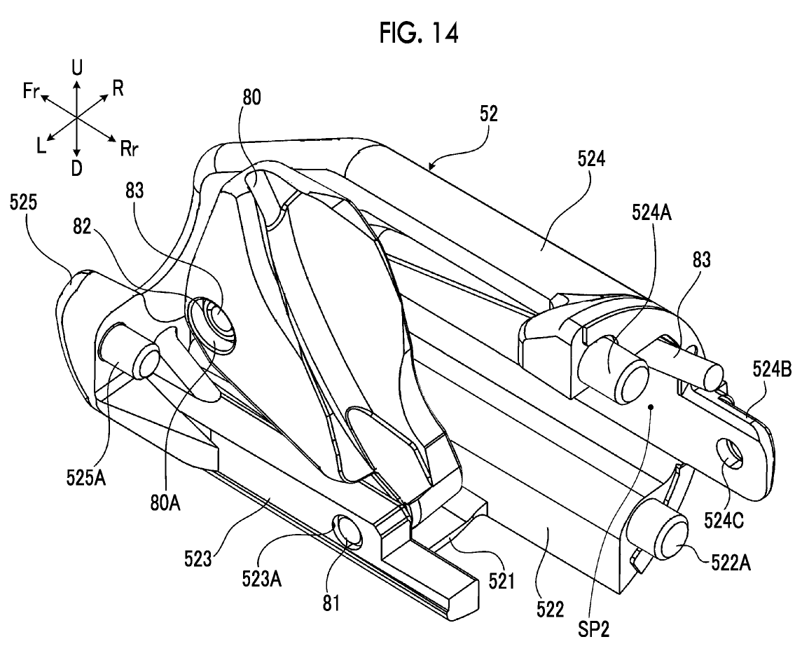

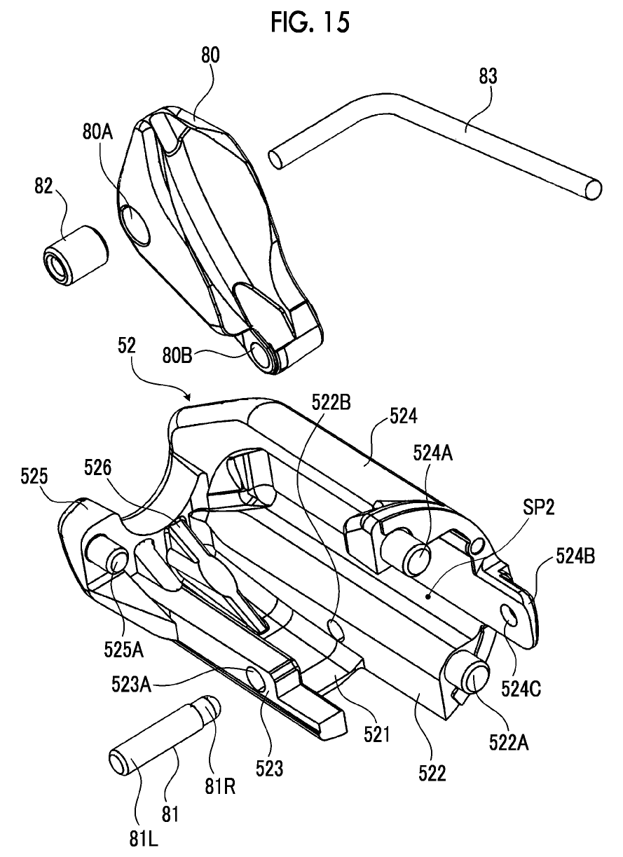

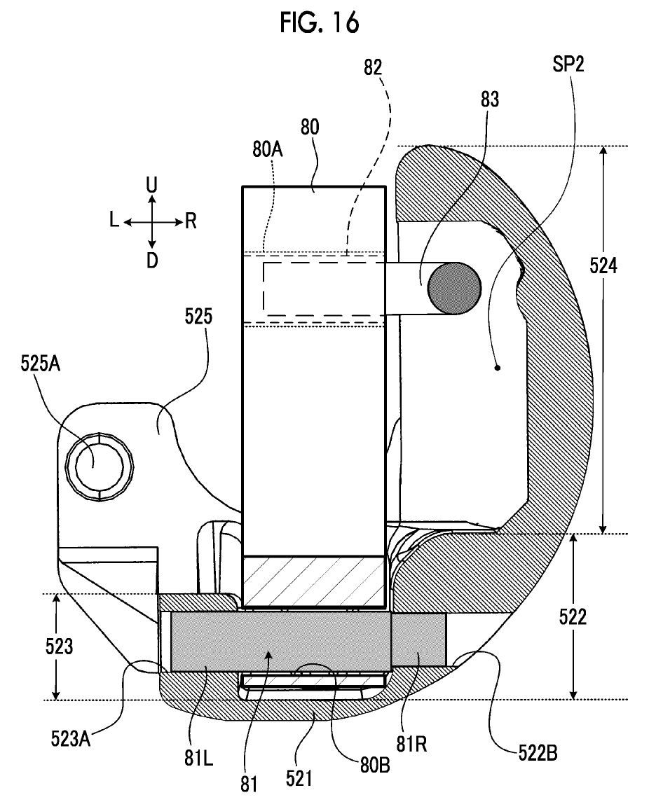

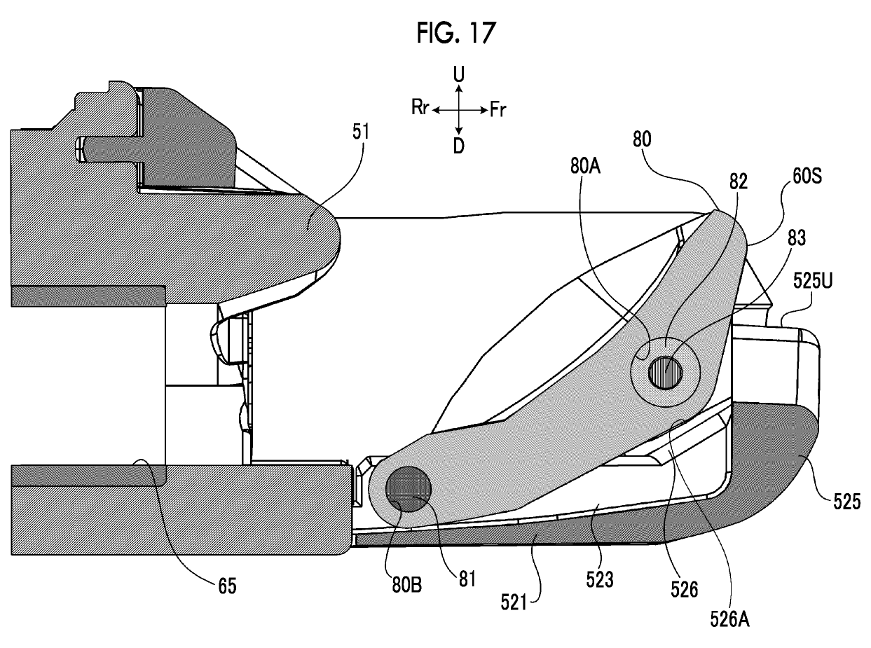

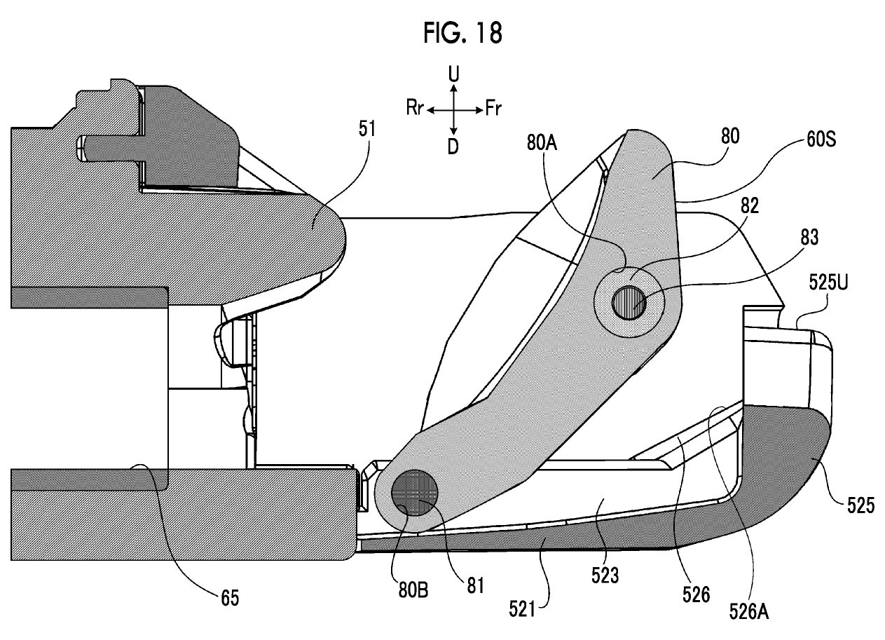

FIG. 14 is a perspective view showing a configuration of the second constituent member 52 and the elevator 80 supported by the second constituent member 52. FIG. 15 is an exploded perspective view of the configuration shown in FIG. 14. FIG. 16 is a schematic view of a cross section taken through a through-hole into which a pivot shaft of the elevator 80 is inserted in FIG. 14. FIG. 17 is a schematic view of a cross section of the distal end portion 44 taken perpendicular to the left-right direction, and is a view showing a state in which the elevator 80 is in a folded position. FIG. 18 is a schematic view of a cross section of the distal end portion 44 taken perpendicular to the left-right direction, and is a view showing a state in which the elevator 80 is in an intermediate upright position.

As shown in FIG. 16, the second constituent member 52 includes a first portion 521 that is disposed on a rear surface side (lower side) of the elevator 80 and that constitutes an outer peripheral surface (lower surface) of the distal end portion 44, and a second portion 522 and a third portion 523 that are provided to extend in the up direction U, which is a movement direction of the elevator 80, from the first portion 521 and that are provided to face each other in the left-right direction, which is a direction intersecting the movement direction of the elevator 80. The second portion 522 is configured to extend upward from the right side of the first portion 521. The third portion 523 is configured to extend upward from the left side of the first portion 521.

The second constituent member 52 further includes a fourth portion 524 that is provided to extend upward from the second portion 522. The first portion 521, the second portion 522, and the fourth portion 524 constitute the outer peripheral surface of the distal end portion 44. The outer peripheral surface of the distal end portion 44 formed by the first portion 521, the second portion 522, and the fourth portion 524 has an arc shape. The fourth portion 524 has a curved shape that bulges to the right side, and defines, on an inner side thereof, a storage space SP2 in which the operation wire 83 can be stored.

As shown in FIG. 15, the fourth portion 524 is provided with an extension piece 524B having a plate shape and a thickness in the left-right direction extending from a proximal end to a proximal end side. The extension piece 524B is provided with a through-hole 524C that extends in the left-right direction.

The extension piece 524B is disposed in the receiving recessed portion 514 of the first constituent member 51 shown in FIG. 9, and the hole portion 514A and the through-hole 524C are disposed to face each other. A fixing pin made of metal is inserted into the hole portion 514A and the through-hole 524C, and the first constituent member 51 and the second constituent member 52 are fixed in a direction orthogonal to the longitudinal direction. The fixing pin may be formed by curing an adhesive injected into the hole portion 514A and the through-hole 524C. The first constituent member 51 and the second constituent member 52 are firmly fixed by the fixing pin.

The second constituent member 52 further includes a fifth portion 525 that is provided on the distal end side of the first portion 521, the second portion 522, and the third portion 523 and that constitutes a distal end surface of the distal end portion 44. The fifth portion 525 is a portion disposed on the distal end side of the third constituent member 53. The fifth portion 525 is provided with the insertion projection portion 525A that constitutes an engagement portion that engages with the third constituent member 53 in the longitudinal direction.

The fifth portion 525 has a C-shape that is open on an upper side as viewed in the longitudinal direction. With this configuration, as shown in FIG. 6, at least a part of a rear surface of the elevator 80 is visible as viewed in the longitudinal direction in a state in which the elevator 80 is in at least one of an upright position or a folded position.

As shown in FIG. 16, the second portion 522 is provided with a through-hole 522B that extends in the left-right direction. The third portion 523 is provided with a through-hole 523A that extends in the left-right direction. The through-hole 522B and the through-hole 523A have the same position in the up-down direction and are disposed below the outlet port 65.

As shown in FIG. 15, the elevator unit includes the elevator 80, a wire fixing hole 80A, which is a through-hole that extends in the left-right direction and that is provided on a distal end side (a side opposite to a pivot shaft side) of the elevator 80, a metal fastener 82 inserted into the wire fixing hole 80A, an operation wire 83 of which a distal end side is inserted into the fastener 82, a pivot shaft hole 80B, which is a through-hole that extends in the left-right direction and that is provided on a proximal end side of the elevator 80, and a columnar pivot shaft 81 that constitutes a shaft member inserted into the pivot shaft hole 80B. A closely attached spring may be used as the fastener 82. The pivot shaft 81 has a stepped structure including a small-diameter portion 81R and a large-diameter portion 81L having a greater outer diameter than the small-diameter portion 81R. Since the pivot shaft 81 is a portion to which a large force is applied, it is preferable that the pivot shaft 81 is made of metal.

An inner diameter of the through-hole 522B is a size into which the small-diameter portion 81R of the pivot shaft 81 is insertable. An inner diameter of the through-hole 523A is a size into which the large-diameter portion 81L of the pivot shaft 81 is insertable. An inner diameter of the pivot shaft hole 80B of the elevator 80 is greater than a diameter of the large-diameter portion 81L of the pivot shaft 81, so that the elevator 80 is pivotable about the pivot shaft 81.

As shown in FIG. 16, the pivot shaft hole 80B of the elevator 80 is disposed between the through-hole 523A and the through-hole 522B, and the pivot shaft 81 is inserted into these three holes. The small-diameter portion 81R of the pivot shaft 81 extends from the pivot shaft hole 80B to the right side and is inserted into the through-hole 522B. The large-diameter portion 81L of the pivot shaft 81 extends from the pivot shaft hole 80B to the left side, is inserted into the through-hole 523A, and is fixed by an adhesive or the like.

In this way, one end side of the pivot shaft 81 is supported by the second portion 522, and the other end side of the pivot shaft 81 is supported by the third portion 523. The through-hole 523A and the through-hole 522B constitute a support portion that supports the pivot shaft 81. The small-diameter portion 81R that extends from the pivot shaft hole 80B to the right side constitutes a first region of the pivot shaft 81, and the large-diameter portion 81L that extends from the pivot shaft hole 80B to the left side constitutes a second region of the pivot shaft 81.

In a state shown in FIG. 17, in a case where the operation wire 83 is pulled to the proximal end side by operation of the operation unit 45, the elevator 80 pivots about the pivot shaft 81, and a distal end thereof moves in the up direction U as shown in FIG. 18. The movement direction of a distal end of the elevator 80 is defined as the movement direction of the elevator 80.

As shown in FIG. 16, a cylindrical fastener 82 is fixed to the wire fixing hole 80A, and the distal end of the operation wire 83 is inserted into and fixed to the fastener 82 from the right side. The operation wire 83 is drawn out from a right-side surface of the elevator 80 and is disposed in the storage space SP2. The operation wire insertion hole port 64 of the first constituent member 51 is disposed in the storage space SP2, and the operation wire 83 is inserted into the operation wire insertion hole port 64.

As shown in FIG. 15, the second constituent member 52 is provided with a projection portion 526 that projects upward from the first portion 521 and that extends to the distal end side. The projection portion 526 is disposed between the second portion 522 and the third portion 523, on a distal end side of the pivot shaft 81 and on a lower side of the elevator 80. The projection portion 526 is configured as a rib having a triangular shape as viewed in the left-right direction and extending across the first portion 521 and the fifth portion 525. As shown in FIG. 18, a surface 526A on the upper side of the projection portion 526 is an inclined surface of which a distal end side is higher than a proximal end side.

In a state shown in FIG. 18, in a case where the operation wire 83 is returned to the distal end side, the elevator 80 pivots about the pivot shaft 81, a distal end thereof moves in the down direction D, and the rear surface (surface on a side opposite to the outlet port 65 side) of the elevator 80 comes into contact with the surface 526A. This state is a folded position of the elevator 80.

Since a force applied to the elevator 80 in a case where the elevator 80 transitions from an upright position to a folded position can be received by the projection portion 526, the durability of the second constituent member 52 on a distal end side can be improved.

Even in a case where a gallstone enters between the elevator 80 and the projection portion 526, the gallstone can be discharged from a recessed portion formed in the fifth portion 525 by moving the elevator 80 to the folded position or by supplying water from the outlet port 65.

The elevator 80 is pulled by the operation wire 83 from the right side. In a case where a pulling force is applied to the operation wire 83, in addition to an elevating force, a rotational force that tends to incline the elevator 80 in a direction in which the operation wire 83 is pulled acts on the elevator 80. These forces also act on the pivot shaft 81, the second portion 522 that supports the pivot shaft 81, and the third portion 523. Since the second portion 522 and the third portion 523 are integrally formed via the first portion 521 and the pivot shaft 81 is supported by the through-hole 523A and the through-hole 522B, the durability can be improved. The first portion 521 is thinner than the other portions. Therefore, by providing the projection portion 526, it is possible to prevent the second constituent member 52 from being deformed by the force applied to the vicinity of the through-hole 522B due to the above-described rotational force. In this way, the durability of the second constituent member 52 can be improved.

In the present embodiment, the pivot shaft 81 is disposed at a position close to an outer surface of the distal end portion 44. For example, in the distal end portion 44, the outlet port 65 is disposed on a lower side (an outer peripheral side of the distal end portion 44), and the pivot shaft 81 is disposed further on the lower side (outer peripheral side of the distal end portion 44) than the outlet port 65. Therefore, the elevator 80 can be made longer. As a result, it is possible to significantly change an orientation of a treatment tool delivered from the outlet port 65.

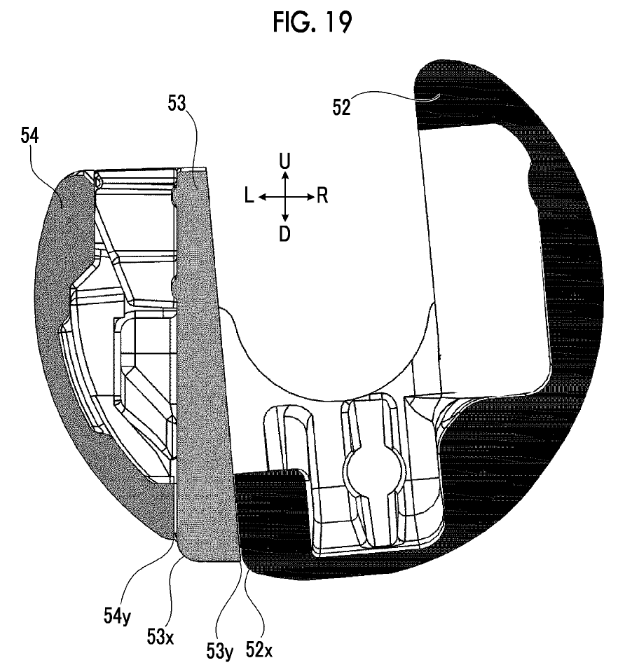

FIG. 19 is a schematic view of a cross section of the distal end portion 44 taken perpendicular to a longitudinal direction thereof. As shown in FIG. 19, an end portion 52x of the second constituent member 52 and an end portion 53y of the third constituent member 53 are disposed close to each other at a boundary portion between the second constituent member 52 and the third constituent member 53.

The end portion 53y of the third constituent member 53 is a portion corresponding to a parting line of a mold in a case where the third constituent member 53 is formed by molding a resin using the mold. The end portion 52x of the second constituent member 52 is a portion corresponding to a part other than the parting line of the mold in a case where the second constituent member 52 is formed by molding a resin using the mold. The end portion 52x has a shape that is more rounded than the end portion 53y. The end portion 52x is disposed more outward than the end portion 53y on the distal end portion 44.

An end portion 53x of the third constituent member 53 and an end portion 54y of the fourth constituent member 54 are disposed close to each other at a boundary portion between the third constituent member 53 and the fourth constituent member 54.

The end portion 53x of the third constituent member 53 is a portion corresponding to a part other than the parting line of the mold in a case where the third constituent member 53 is formed by molding a resin using the mold. The end portion 54y of the fourth constituent member 54 is a portion corresponding to the parting line of the mold in a case where the fourth constituent member 54 is formed by molding a resin using the mold. The end portion 53x has a shape that is more rounded than the end portion 54y. The end portion 53x is disposed more outward than he end portion 54y on the distal end portion 44.

As shown in FIG. 6, similarly at the boundary portion between the first constituent member 51 and the second constituent member 52, a portion corresponding to a part other than a parting line of one of the constituent members is disposed more outward than a portion corresponding to a parting line of the other constituent member.

With such a configuration, since an angular portion corresponding to the parting line is disposed inward of a non-angular portion, it is possible to prevent the angular portion from coming into contact with a subject. By allowing the presence of the angular portion corresponding to the parting line, it is possible to reduce the manufacturing costs of the distal end portion 44. Among the four members constituting the distal end portion 44, one of two members adjacent to each other constitutes the first distal end member, and the other constitutes the second distal end member.

By configuring the single-use endoscope 40 as described above, the outer diameter of the distal end portion 44 can be set to 7.0 mm or more and 10.3 mm or less, the outer diameter of the insertion part 41 can be set to 7.4 mm or more and 10.3 mm or less, and the strength of the distal end portion 44 can be set to a level that can withstand practical use (for example, sufficient to withstand tension of the operation wire 83 during an elevating operation of the elevator 80). In addition, a ratio of a length of the elevator 80 to the outer diameter of the distal end portion 44 can be set to 0.91 or more.

The outer diameter of the distal end portion 44 is defined as a diameter of a perfect circle circumscribing all the members (the first constituent member 51, the second constituent member 52, the third constituent member 53, and the fourth constituent member 54) constituting the distal end portion 44 in a case where the distal end portion 44 is viewed in the longitudinal direction.

The outer diameter of the insertion part 41 is defined as a diameter of a perfect circle circumscribing the thickest portion of the insertion part 41 in a case where the insertion part 41 is viewed in the longitudinal direction.

The length of the elevator 80 is defined by a distance from the pivot shaft 81 to the distal end thereof.

In the above description, the single-use endoscope 40 has been described as being for single use, but may be configured to be usable multiple times.

As described so far, at least the following matters are described in the present specification.

(1)
An endoscope comprising:
an insertion part that is to be inserted into a subject;
a distal end portion that is disposed on a distal end side of the insertion part; and
an elevator for a treatment tool that is disposed on the distal end portion,
in which an outer diameter of the distal end portion is 7.0 mm or more and 10.3 mm or less, and
an outer diameter of the insertion part is 7.4 mm or more and 10.3 mm or less.

(2)
The endoscope according to (1),
in which a ratio of a length of the elevator to the outer diameter of the distal end portion is 0.91 or more.

(3)
The endoscope according to (1) or (2),
in which the elevator and the distal end portion are made to contain a resin material.

(4)
The endoscope according to any one of (1) to (3), further comprising:
an illumination portion that is disposed on an outer peripheral side of the distal end portion and that emits illumination light; and
an observation portion that is disposed on the outer peripheral side of the distal end portion and on which observation light obtained by reflection of the illumination light by the subject is incident.

(5)
The endoscope according to any one of (1) to (4), further comprising:
a bendable part that is disposed on a proximal end side of the distal end portion in the insertion part and that is capable of performing a bending operation;
a tubular body that is provided in the bendable part and that is formed of a plurality of annular members rotatably connected to each other; and
a covering portion that is provided in the bendable part and that directly covers the tubular body.

(6)
The endoscope according to any one of (1) to (5),
in which the endoscope is for single use.

(7)
The endoscope according to any one of (1) to (6),
in which the distal end portion includes a plurality of members.

(8)
The endoscope according to (7),
in which the plurality of members have engagement portions that engage with each other in a longitudinal direction of the insertion part.

(9)
The endoscope according to (7) or (8),
in which at least one of the plurality of members is configured to be radiopaque.

(10)
The endoscope according to any one of (7) to (9),
in which one of the plurality of members includes a first portion that is disposed on a rear surface side of the elevator and that constitutes an outer peripheral surface of the distal end portion, and a second portion and a third portion that are provided to extend from the first portion in a movement direction of the elevator and that are provided to face each other in a direction intersecting the movement direction,
the endoscope further comprises:
a through-hole that is provided on a proximal end side of the elevator and that extends in the intersecting direction; and
a shaft member that is inserted into the through-hole,
one end side of the shaft member is supported by the second portion, and
the other end side of the shaft member is supported by the third portion.

(11)
The endoscope according to (10),
in which the elevator and the distal end portion are made to contain a resin material, and
the shaft member is made to contain metal.

(12)
The endoscope according to any one of (7) to (11),
in which the plurality of members include a first distal end member and a second distal end member that constitute an outer peripheral surface of the distal end portion,
the first distal end member includes a first end portion that constitutes the outer peripheral surface and that is provided at a boundary portion with the second distal end member,
the second distal end member includes a second end portion that constitutes the outer peripheral surface and that is provided at a boundary portion with the first distal end member, the second end portion being more rounded than the first end portion, and
the second end portion is disposed more outward than the first end portion on the distal end portion.

[1]
An endoscope comprising:
an insertion part that is to be inserted into a subject;
a distal end portion that is disposed on a distal end side of the insertion part and that includes a plurality of members;
an elevator for a treatment tool that is disposed in the distal end portion;
a through-hole that is provided on a proximal end side of the elevator and that extends in a direction intersecting a movement direction of the elevator as viewed in a longitudinal direction of the insertion part; and
a shaft member that is inserted into the through-hole,
in which a first member, which is one of the plurality of members, includes: a first portion disposed on a rear surface side of the elevator and constituting an outer peripheral surface of the distal end portion; and a second portion and a third portion that extend from the first portion in the movement direction and are provided to face each other in a direction intersecting the movement direction,
one end side of the shaft member is supported by the second portion, and
the other end side of the shaft member is supported by the third portion.

[2]
The endoscope according to [1],
in which the first member includes a fourth portion that is provided to extend from one of the second portion and the third portion in the movement direction and that constitutes the outer peripheral surface of the distal end portion.

[3]
The endoscope according to [2],
in which the first member includes a fifth portion that is provided on distal end sides of the first portion, the second portion, and the third portion and that constitutes a distal end surface of the distal end portion.

[4]
The endoscope according to [3],
in which the fifth portion of the first member is configured such that at least a part of a rear surface of the elevator is visually recognizable as viewed in the longitudinal direction of the insertion part.

[5]
The endoscope according to any one of [1] to [4],
in which the first member includes a projection portion that is disposed between the second portion and the third portion, that protrudes in the movement direction, and that extends toward a distal end side, and
a surface of the projection portion in the movement direction is an inclined surface having a distal end side higher than a proximal end side.

[6]
The endoscope according to [5],
in which, in a case where the elevator is in a folded state, a rear surface of the elevator contacts the inclined surface.

[7]
The endoscope according to any one of [1] to [6],
in which the shaft member includes a first region that protrudes from the through-hole of the elevator to one side, and a second region that protrudes from the through-hole of the elevator to the other side and that has a greater outer diameter than the first region,
the first region is inserted into a hole portion provided in the second portion, and
the second region is inserted into a hole portion provided in the third portion.

[8]
The endoscope according to any one of [1] to [7], further comprising:
an operation wire that is connected to a distal end side of the elevator,
in which the operation wire is drawn out from a side surface of the elevator in an axial direction of the shaft member.

[9]
The endoscope according to [8],
in which the first member includes a fourth portion that is provided to extend from one of the second portion and the third portion in the movement direction, that constitutes the outer peripheral surface of the distal end portion, and that defines a storage space that stores the operation wire.

<1>
An endoscope comprising:
an insertion part that is to be inserted into a subject; and
a distal end portion that is disposed on a distal end side of the insertion part and that includes a plurality of members,
in which the plurality of members include engagement portions that engage with each other in a longitudinal direction of the insertion part.

<2>
The endoscope according to <1>,
in which the plurality of members include a first constituent member, a second constituent member disposed on a distal end side with respect to the first constituent member, and a third constituent member disposed on the distal end side of the first constituent member, and
the engagement portions include a first engagement portion that engages the first constituent member and the second constituent member, and a second engagement portion that engages the first constituent member and the third constituent member.

<3>
The endoscope according to <2>,
in which the plurality of members include a fourth constituent member disposed on an outer peripheral side of the distal end portion of the third constituent member, and
the fourth constituent member and the third constituent member include engagement portions that engage with each other in a direction orthogonal to the longitudinal direction.

<4>
The endoscope according to <2> or <3>,
in which the second constituent member includes a portion disposed on a distal end side of the third constituent member, and
the engagement portions include a third engagement portion that engages the portion and the third constituent member.

<5>
The endoscope according to any one of <2> to <4>,
in which the engagement portions are formed by a combination of a hole portion and an insertion projection portion inserted into the hole portion.

<6>
The endoscope according to any one of <2> to <5>, further comprising:
a treatment tool outlet port provided in the first constituent member.

<7>
The endoscope according to any one of <2> to <6>, further comprising:
an elevator for a treatment tool disposed in the distal end portion; and
a support portion that is provided in the second constituent member and that supports a pivot shaft of the elevator.

<8>
The endoscope according to any one of <2> to <7>, further comprising:
an illumination portion that is provided in the third constituent member and that emits illumination light; and
an observation portion that is provided in the third constituent member and on which observation light obtained by reflection of the illumination light by the subject is incident.

<9>
The endoscope according to <8>, further comprising:
an imaging element that images the subject through the observation portion and a light emitting element that generates the illumination light, the imaging element and the light emitting element being provided at the distal end portion,
in which the plurality of members include a fourth constituent member that is disposed on an outer peripheral side of the distal end portion of the third constituent member, and
a substrate that is connected to the imaging element and signal lines that are connected to the substrate and to the light emitting element are disposed between the third constituent member and the fourth constituent member.

<10>
The endoscope according to <9>,
in which the fourth constituent member constitutes an outer peripheral surface of the distal end portion.

<11>
The endoscope according to <9> or <10>,
in which the fourth constituent member includes an extension region that extends from a proximal end toward a proximal end side and that overlaps the first constituent member in a direction perpendicular to the longitudinal direction of the insertion part, and
the signal lines are disposed between the extension region and the first constituent member.

[1]
An endoscope comprising:
an insertion part that is to be inserted into a subject; and
a distal end portion that is disposed on a distal end side of the insertion part and that includes a plurality of members,
in which the plurality of members include a first distal end member and a second distal end member that constitute an outer peripheral surface of the distal end portion,
the first distal end member includes a first end portion that constitutes the outer peripheral surface and that is provided at a boundary portion with the second distal end member,
the second distal end member includes a second end portion that constitutes the outer peripheral surface and that is provided at a boundary portion with the first distal end member, the second end portion being more rounded than the first end portion, and
the second end portion is disposed more outward than the first end portion on the distal end portion.

[2]
The endoscope according to [1],
in which the first distal end member is formed by resin molding using a mold, and
the first end portion corresponds to a parting line of the mold.

[3]
The endoscope according to [1] or [2],
in which the second distal end member is formed by resin molding using a mold, and
the second end portion corresponds to a part other than the parting line of the mold.

### EXPLANATION OF REFERENCES

13: first support member
13A, 14A: outer peripheral surface
14: second support member
40: single-use endoscope
41: insertion part
42: flexible part
43: bendable part
44: distal end portion
44A: observation portion
44B: illumination portion
45: operation unit
46: first cord part
46A, 47A: connector
47: second cord part
50: adapter
51: first constituent member
52: second constituent member
52x, 53x, 53y, 54y: end portion
53: third constituent member
53A, 53B: recessed portion
53C, 53D, 54A, 54B, 54C, 522A, 524A, 525A: insertion projection portion
53E, 53F, 53G, 516, 517, 518, 519, 520: engagement hole portion
54: fourth constituent member
54D: extension region
63: air/water supply port
64: operation wire insertion hole port
65: outlet port
71: light emitting unit
72: imaging optical system
73: imaging element
74: substrate
75, 76, 471: signal line
80: elevator
80A: wire fixing hole
80B: pivot shaft hole
81: pivot shaft
81L: large-diameter portion
81R: small-diameter portion
82: fastener
83: operation wire
83A: wire covering member
100: endoscope device
430: structure
431: bending piece
432: covering member
440: accommodation recessed portion
450: operation unit pipe line
451: treatment tool pipe line
452: branch member
453: treatment tool inlet port
454, 456: valve mechanism
455: suction nozzle
461: air/water supply pipe line
461A: air/water supply connection fitting
510: base
510A: distal end-side protruding portion
510B: proximal end-side protruding portion
511, 512, 515: cutout
513, 522B, 523A, 524C: through-hole
514: receiving recessed portion
514A: hole portion
521: first portion
522: second portion
523: third portion
524: fourth portion
524B: extension piece
525: fifth portion
526: projection portion
526A: surface
530: upper surface wall portion
531: distal end wall portion
800: control device
800A: operation lever
801A: connector connection portion
801B: input device
900: display device
SP1: space
SP2: storage space

What is claimed is:

1. An endoscope comprising:
an insertion part that is to be inserted into a subject;
a distal end portion that is disposed on a distal end side of the insertion part; and
an elevator for a treatment tool that is disposed on the distal end portion,
wherein an outer diameter of the distal end portion is 7.0 mm or more and 10.3 mm or less, and
an outer diameter of the insertion part is 7.4 mm or more and 10.3 mm or less.

2. The endoscope according to claim 1,
wherein a ratio of a length of the elevator to the outer diameter of the distal end portion is 0.91 or more.

3. The endoscope according to claim 1,
wherein the elevator and the distal end portion are made to contain a resin material.

4. The endoscope according to claim 1, further comprising:
an illumination portion that is disposed on an outer peripheral side of the distal end portion and that emits illumination light; and
an observation portion that is disposed on the outer peripheral side of the distal end portion and on which observation light obtained by reflection of the illumination light by the subject is incident.

5. The endoscope according to claim 2, further comprising:
an illumination portion that is disposed on an outer peripheral side of the distal end portion and that emits illumination light; and
an observation portion that is disposed on the outer peripheral side of the distal end portion and on which observation light obtained by reflection of the illumination light by the subject is incident.

6. The endoscope according to claim 3, further comprising:
an illumination portion that is disposed on an outer peripheral side of the distal end portion and that emits illumination light; and
an observation portion that is disposed on the outer peripheral side of the distal end portion and on which observation light obtained by reflection of the illumination light by the subject is incident.

7. The endoscope according to claim 1, further comprising:
a bendable part that is disposed on a proximal end side of the distal end portion in the insertion part and that is capable of performing a bending operation;
a tubular body that is provided in the bendable part and that is formed of a plurality of annular members rotatably connected to each other; and
a covering portion that is provided in the bendable part and that directly covers the tubular body.

8. The endoscope according to claim 2, further comprising:
a bendable part that is disposed on a proximal end side of the distal end portion in the insertion part and that is capable of performing a bending operation;
a tubular body that is provided in the bendable part and that is formed of a plurality of annular members rotatably connected to each other; and
a covering portion that is provided in the bendable part and that directly covers the tubular body.

9. The endoscope according to claim 3, further comprising:
a bendable part that is disposed on a proximal end side of the distal end portion in the insertion part and that is capable of performing a bending operation;
a tubular body that is provided in the bendable part and that is formed of a plurality of annular members rotatably connected to each other; and
a covering portion that is provided in the bendable part and that directly covers the tubular body.

10. The endoscope according to claim 1,
wherein the endoscope is for single use.

11. The endoscope according to claim 2,
wherein the endoscope is for single use.

12. The endoscope according to claim 3,
wherein the endoscope is for single use.

13. The endoscope according to claim 1,
wherein the distal end portion includes a plurality of members.

14. The endoscope according to claim 2,
wherein the distal end portion includes a plurality of members.

15. The endoscope according to claim 3,
wherein the distal end portion includes a plurality of members.

16. The endoscope according to claim 13,
wherein the plurality of members have engagement portions that engage with each other in a longitudinal direction of the insertion part.

17. The endoscope according to claim 13,
wherein at least one of the plurality of members is configured to be radiopaque.

18. The endoscope according to claim 13,
wherein one of the plurality of members includes a first portion that is disposed on a rear surface side of the elevator and that constitutes an outer peripheral surface of the distal end portion, and a second portion and a third portion that are provided to extend from the first portion in a movement direction of the elevator and that are provided to face each other in a direction intersecting the movement direction,
the endoscope further comprises:
a through-hole that is provided on a proximal end side of the elevator and that extends in the intersecting direction; and
a shaft member that is inserted into the through-hole,
one end side of the shaft member is supported by the second portion, and
other end side of the shaft member is supported by the third portion.

19. The endoscope according to claim 18,
wherein the elevator and the distal end portion are made to contain a resin material, and
the shaft member is made to contain metal.

20. The endoscope according to claim 13,
wherein the plurality of members include a first distal end member and a second distal end member that constitute an outer peripheral surface of the distal end portion,
the first distal end member includes a first end portion that constitutes the outer peripheral surface and that is provided at a boundary portion with the second distal end member,
the second distal end member includes a second end portion that constitutes the outer peripheral surface and that is provided at a boundary portion with the first distal end member, the second end portion being more rounded than the first end portion, and
the second end portion is disposed more outward than the first end portion on the distal end portion.
